(19) United States
(12) Patent Application Publication
(10) Pub. No.: US 2006/0204537 A1
(43) Pub. Date: Sep. 14, 2006

(54) Silicone blends and composites for drug delivery

(75) Inventors: Buddy Ratner, Seattle, WA (US); Connie Kwok, Seattle, WA (US); Katie Walline, Boulder, CO (US); Erika Johnston, Cambridge, MA (US); Robert Miller, Cambridge, MA (US)

Correspondence Address: COHEN, PONTANI, LIEBERMAN & PAVANE, 551 FIFTH AVENUE, SUITE 1210, NEW YORK, NY, 10176, US

(73) Assignee: Genzyme Corporation

(21) Appl. No.: 10/518,562

(22) PCT Filed: Jun. 20, 2003
(86) PCT No.: PCT/US03/19676

§ 371 Date: Nov. 17, 2005

Related U.S. Application Data
(60) Provisional application No. 60/390,665, filed on Jun. 21, 2002.

(51) Int. Cl. A61K 48/00; A61K 31/4745; A61F 2/02

(52) U.S. Cl. 424/423; 514/44; 514/291

(57) ABSTRACT

The present invention provides a composition for use in delivering a drug into the body of a mammal, wherein the composition comprises silicone elastomer, an adjuvant polymer, and the drug. This composition may be part of an implantable medical device, such as a stent or a vascular or other graft or sheath, among other configurations. When the compositions are used as coating, the coating may further include a topcoat of silicone or silicone and adjuvant polymer mixture.

### BACKGROUND OF THE INVENTION

1. Field of the Invention

The present invention relates to implantable medical devices for the controlled, localized delivery of bioactive drugs within a body.

2. Description of the Related Art

The systemic administration of drug agents, such as by intravenous means, treats the body as a whole even though the disease to be treated may be localized. Thus, it has become common to treat a variety of medical conditions by introducing an implantable medical device partly or completely into a body cavity such as the esophagus, trachea, colon, biliary tract, urinary tract, vascular system or other location within a human or veterinary patient.

For example, many treatments of the vascular system entail the introduction of a device such as a stent, catheter, balloon, guide wire, cannula or the like. One of the potential drawbacks to conventional drug delivery techniques with the use of these devices being introduced into and manipulated through the vascular system is that blood vessel walls can be disturbed or injured. Clot formation or thrombosis often results at the injured site, causing stenosis (closure) of the blood vessel.

Another cause of stenosis is vascular disease. Probably the most common disease causing stenosis of blood vessels is atherosclerosis. Atherosclerosis is a condition which commonly affects the coronary arteries, the aorta, the iliofemoral arteries and the carotid arteries.

Many medical devices and therapeutic methods are known for the treatment of atherosclerotic disease. One particular therapy for certain atherosclerotic lesions is percutaneous transluminal coronary revascularization (PTCR), which is a widely performed procedure used to open coronary arteries that have been blocked due to atherosclerotic plaque. PTCR is done most commonly via balloon angioplasty, where a small balloon is threaded into the blocked artery and inflated. Inflation of the balloon “cracks” the atherosclerotic plaque and expands the vessel, thereby relieving the stenosis, at least in part.

PTCR is performed more than two million times annually worldwide. While PTCR presently enjoys wide use, it suffers from two major problems. First, the blood vessel may suffer acute occlusion immediately after or within the initial hour after the dilation procedure. Such occlusion is referred to as “abrupt closure.” A second major problem encountered in PTCR is the re-narrowing of an artery after an initially successful angioplasty. This re-narrowing is referred to as “restenosis” and typically occurs within the first six months after angioplasty. Restenosis is believed to arise through the proliferation and migration of cellular components from the arterial wall, as well as through geometric changes in the arterial wall referred to as “remodeling.”

A device such as an intravascular stent including stent grafts and covered stents can be a useful adjunct to PTCR, particularly in the case of either acute or threatened closure after angioplasty. The stent is placed in the dilated segment of the artery to mechanically prevent abrupt closure and restenosis.

Unfortunately, even when the implantation of the stent is accompanied by aggressive and precise antiplatelet and anticoagulation therapy (typically by systemic administration), the incident of thrombotic vessel closure or other thrombotic complication remains significant, and the prevention of restenosis is not as successful as desired. Restenosis occurs in 30-40% of patients without stents and in 15-30% of patients receiving stents. However, an undesirable side effect of the systemic antiplatelet and anticoagulation therapy is an increased incidence of bleeding complications, most often at the percutaneous entry site.

Other conditions and diseases are also treatable with stents, catheters, cannulae and other devices inserted into the esophagus, trachea, colon, biliary tract, urinary tract and other locations in the body, or with orthopedic devices, implants, or replacements, for example. Unfortunately, bacterial infections are often observed with prosthetic implants and in many cases result in the failure of the devices. Bacteria have a remarkable ability to adhere to surfaces and form biofilms. If they attach to medical implants and cause infection, this phenomenon is referred to as device-associated or biofilm-related infection. Once formed, a biofilm is extremely difficult to eradicate, even with vigorous antibiotic treatments. One object of the present invention is to provide implantable medical devices coated with a layer containing an antibiotic that would be released in a controlled manner to prevent bacterial colonization and biofilm formation.

One of the drawbacks of conventional means of drug delivery using such coated medical devices is the difficulty in effectively delivering the bioactive agent over a short term (that is, the initial hours and days after insertion of the device) as well as over a long term (the weeks and months after insertion of the device). Another difficulty with the conventional use of stents for drug delivery purposes is providing precise control over the delivery rate of the desired bioactive agents, drug agents or other bioactive material. The term “bioactive agent” is used herein to mean any agent such as a pharmaceutical agent or drug or other material that has a therapeutic effect.

It is desirable to develop devices and methods for reliably delivering suitable amounts of therapeutic agents, drugs or bioactive materials directly into a body portion during or following a medical procedure, so as to treat or prevent such conditions and diseases, for example, to prevent abrupt closure and/or restenosis of a body portion such as a passage, lumen or blood vessel or to prevent bacterial infection.

In view of the potential drawbacks to conventional drug delivery techniques, there exists a need for a device, method and method of manufacture which enable a controlled localized delivery of active agents, drug agents or bioactive material to target locations within a body.

### SUMMARY OF THE INVENTION

The foregoing problems are solved and a technical advance is achieved in an illustrative cardiovascular stent or other implantable medical device that provides a controlled release of at least one bioactive agent into the vascular or other system, or other location in the body, into which the stent or medical device is positioned. In one aspect, the present invention provides a composition comprising a blend of silicone elastomer, an adjuvant polymer and a drug for the controlled release of the drug. The composition can be used to form medical devices in part such as a coating or in their entirety. In another aspect, the invention provides for medical devices made in parts or in their entirety from the composition of the invention.

The various features of novelty which characterize the invention are pointed out with particularity in the claims annexed to and forming a part of the disclosure. For a better understanding of the invention, its operating advantages, and specific objects attained by its use, reference should be had to the drawings and descriptive matter illustrated therein and preferred embodiments of the invention set forth below.

### BRIEF DESCRIPTION OF THE DRAWINGS

In the drawings:

FIG. 1 is a comparison of release of Paclitaxel from silicone elastomer coatings made with and without 20% PEG into calf serum;

FIG. 2 is a comparison of release of Tranilast from silicone elastomer coatings made with and without 20% PEG into PBS, pH=7.4;

FIG. 3 is a comparison of release into PBS, pH=7.4 of Tranilast from silicone elastomer coatings with topcoats made of silicone and silicone/PEG;

FIG. 4 shows a percentage of methylene blue released;

FIG. 5 shows a percentage of methylene blue released-reduced scale;

FIG. 6 shows a release rate of methylene blue after one week (r2=0.88-0.98);

FIG. 7 shows the amount of DENSPM in test disks as a function of disk composition;

FIG. 8 shows deformation scores of DENSPM-loaded silicone composites after two weeks soaking in phosphate buffered saline at 37° C., n=3;

FIG. 9 shows the Young's modulus (MPa) of DENSPM-loaded films prior to gamma irradiation, as a function of composition, n=3;

FIG. 10 shows the Young's modulus (MPa) of DENSPM-loaded films after 2.6 MRad gamma irradiation, as a function of composition, n=3;

FIG. 11 shows the Young's modulus (MPa) of gamma irradiated, DENSPM-loaded films after two weeks soaking, as a function of composition, n=3; and

FIG. 12 shows Kinetic release of DENSPM from PDMS-DENSPM-PEG composites.

### DETAILED DESCRIPTION OF THE PRESENTLY PREFERRED EMBODIMENTS

The present invention provides silicone composites (also referred to herein as blends) that are suitable for use as controlled drug delivery as coatings of stents and other implantable devices for example, or as bulk material to form portions or the entirety of implantable medical devices. Hydrophobic molecules can be delivered directly from silicone composites and the rate of elution modulated by the addition of one or more adjuvant polymers. The initial burst of hydrophilic molecules from the silicone composites is greatly reduced by the presence of the adjuvant polymer and the subsequent release rate can be controlled by the properties of the adjuvant polymer. We have demonstrated that smoother, more uniform composite films can be formed or cast by deposition from solutions prepared from mixtures of solvents and that certain medical devices such as sheaths that are suitable for drug delivery can be formed entirely from these solutions.

Illustrative adjuvant polymers include polyethylene glycol (PEG) having a molecular weight preferably of about 2 KDa to 1 MDa and most preferably about 2-500 KDa, copolymers of ethylene oxide and propylene oxide (EO/PO) such as Pluronic® polymers which exhibit surfactant properties, as exemplified below, as well as any other hydrophilic polymers, including, but not limited to, polysaccharides such a hyaluronic acid and chemically modified cellulose, polyamyloses, polydextroses, dextrans, heparins, heparans, chondroitin sulfate, dermatan sulfate, poly(N-isopropylacrylamide), polyurethanes, polyacrylates, polyethyleneimines, polyvinylpyrrolidone, polyvinylalcohol, polyvinylacetate, etc. The therapeutics envisioned for delivery include, but are not limited to: antiproliferatives, anti-inflammatories, antibiotics, antiplatelet agents, anticoagulants, antimicrobials, anti-arrhythmic, antisense therapeutics, and genetic material. The coatings can be used to deliver therapeutics from stents, stent grafts, PICC lines, catheters, arterial-venous shunts, artery and vein grafts, urological catheters or stents and any other implantable medical devices from which local therapeutic delivery would be beneficial.

The present invention further provides implantable medical devices and methods for the controlled, localized delivery of a bioactive agent to targeted locations within a body. The term “controlled localized delivery” as used herein is defined as a characteristic profile release rate of the bioactive agent over a desired period of time at a fixed location. The implantable medical devices of the present invention may have a simple construction, provide a minimal cross-sectional profile, and allow for easy and reproducible loading of active agents, drug agents and bioactive material.

### EXAMPLE 1

Paclitaxel is a lipophilic drug that has been shown to prevent restenosis both with oral administration (Sollott (1995), J. Clin. Invest., 95: 1869-1876) and local delivery (Axel (1997), Circulation, 96: 636-645, Herdeg (1998), Semin. Intervent. Cardiol., 3: 197-199, Herdeg (2000), Z Kardol., 89: 390-397 (abstract), Honda (2001), Circulation, 104: 380-383, Farb (2001), Circulation, 104: 473-479, Drachman (2000), J. Am. Coll. Cardiol., 36: 2325-2332. Paclitaxel prevents the proliferation of human arterial smooth muscle cells by shifting the balance of microtubule assembly and disassembly towards assembly, thus producing extremely stable unorganized microtubules inside the cytoplasm. Cell replication is thus inhibited in the G₀/G₁ and late G₂ and/or M phases of the cell cycle Axel (1997) Circulation, 96: 636-645, Schiff (1979), Nature, 277: 665-667). Paclitaxel is a highly lipophilic drug, making it a perfect candidate for local delivery because it can easily pass through the hydrophobic barrier of cell membranes leading to rapid cellular uptake. This property of paclitaxel leads to long-lasting effects, even with small doses.

Tranilast is a hydrophilic drug that has been shown to inhibit migration and proliferation of vascular smooth muscle cells as well as collagen synthesis by these cells (Tamai (1999), Am Heart J, 138: 968-975; Fukuyama (1996), Can. J. Physiol. Pharmacol., 74: 80-84; Kikuchi (1996), European Journal of Pharmacology, 295: 221-227). Several clinical trials have shown that oral administration of Tranilast reduces restenosis rates in patients after PCTA (Tamai (1999) Am Heart J, 138: 968-975, Holmes D (2000) Am. Heart J, 139: 23-31). Local delivery of this drug can allow greater concentration of the drug to reach the artery without increasing systemic plasma levels.

### Experimental Methods

All samples were dip-coated pieces of 316 L stainless steel, measuring 1 cm×1 cm with 26 gauge thickness. The stainless steel pieces were first cleaned by sonicating in 3% Isopanasol, deionised (DI) water, and acetone, each for 6 minutes, then dipped into the silicone composite solutions of the invention. Silicone elastomer solutions were made with DAP® 100% silicone rubber adhesive (from DAP, Inc., Maryland) co-dissolved with either Paclitaxel or Tranilast and 20% (w/w) of polyethylene glycol, MW 3400 (PEG) in methylene chloride. Upon setting, the silicone elastomer solutions formed a film on the steel pieces having a certain thickness. Paclitaxel was loaded at 2% of silicone weight. Tranilast was loaded at 5% of silicone weight. Drug loadings of 100-200 μg/sample were achieved for Paclitaxel and 300-350 μg/sample were achieved for Tranilast. Some samples had a topcoat of silicone elastomer alone. Other samples had a topcoat with PEG to reduce initial burst of drug. All samples were placed in glass culture tubes with 2 mL of either PBS, pH 7.4 or calf serum. The culture tubes were then placed in a shaking water bath at 37° C. and 120 rpm. At each time interval, all of the release media was removed and replaced by fresh solution. Paclitaxel samples were assayed using a competitive inhibition enzyme immunoassay (CIEIA) kit from Hawaii Biotech, Aiea, Hi. Tranilast samples were assayed using UV spectrophotometry at a wavelength of 340 nm.

### Results and Discussion

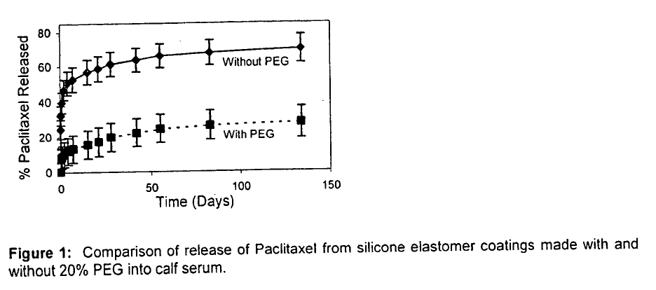

FIG. 1 compares the release of Paclitaxel from films deposited on the stainless steel pieces made both with and without 20% weight polyethylene glycol, MW 3400. As can be seen in FIG. 1, films without PEG have a higher initial burst. Films with PEG, however, have a higher steady state release rate, at 0.38±0.03 μg/day vs. 0.21±0.03 μg/day for films without PEG. Both films exhibit almost zero-order release after the initial burst for the first 60 days, at which point the release rate starts to level off.

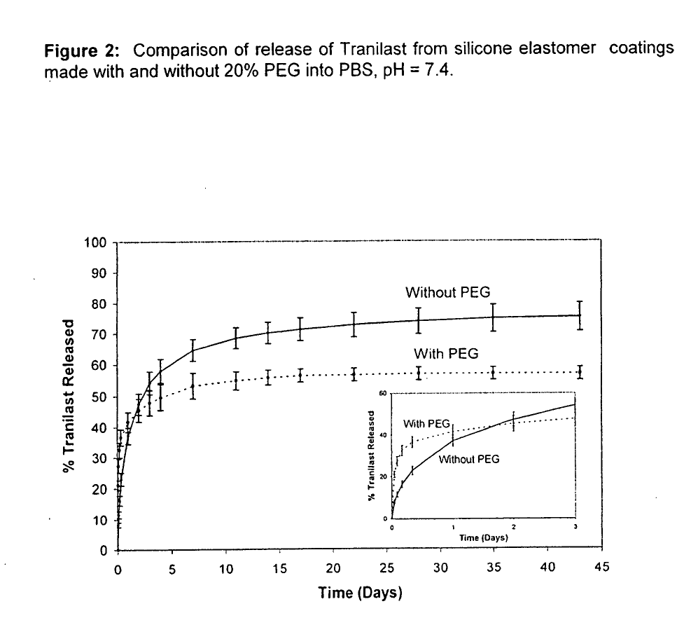

FIG. 2 compares release of Tranilast from films made both with and without about 20% wt PEG. The films with PEG showed a higher initial release rate, which leveled off to a release rate of zero faster than those without PEG.

To slow the rate of the initial burst and extend the release of Tranilast, topcoats of silicone elastomer and silicone with PEG were added. Topcoats were made with either about 1% or about 10% silicone solutions in a non-polar solvent such as toluene with or without % wt. PEG, resulting in topcoats of various thicknesses. For example, to a solution of 1 g silicone and 9 g dimethylene chloride (DMC) is added 0.2 g PEG. The final concentration of PEG in the topcoat is about 16.7%.

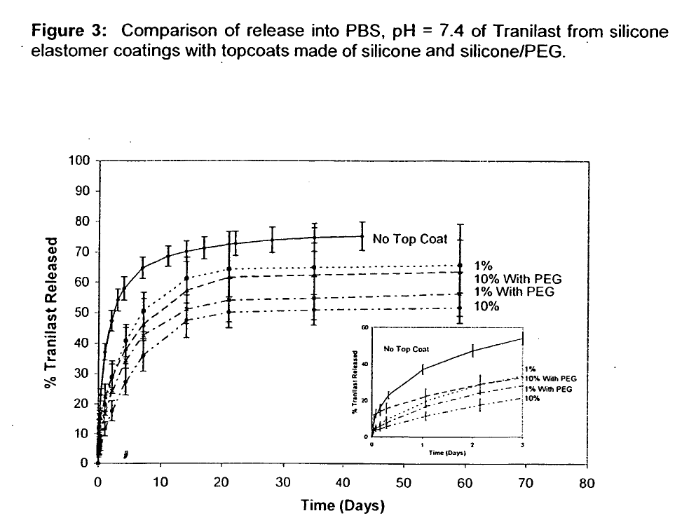

FIG. 3 compares the release into PBS, pH=7.4 of Tranilast from silicone elastomer coatings with topcoats made of silicone and silicone/PEG as described above. As can be seen in FIG. 3, all of the topcoats reduced the initial burst and lengthened the release time of Tranilast. The silicone topcoat made from the 1% solution without PEG allowed the highest fraction of Tranilast to be released. The silicone elastomer topcoat made from the 10% solution without PEG decreased the initial burst rate the most. In all of these samples, the release leveled off after about 21 days.

For the hydrophobic drug Paclitaxel, the incorporation of PEG into silicone elastomer coatings decreases the initial burst rate and raises the steady state release rate of the drug. Near zero-order release rates were achieved for Paclitaxel after the initial burst for 60 days, with continued but decreased release continuing for as long as 140 days.

For a hydrophilic drug, Tranilast, it was shown that the incorporation of PEG increases the initial burst rate while decreasing the subsequent steady state release rate. Release of the drug was not zero order and leveled off to zero after 21 days. Adding a topcoat to the Tranilast/silicone coating somewhat leveled off the initial burst, but did not extend the release past 21 days.

### EXAMPLE 2

Comparison of Dye Release from Silicones Containing a Range of Adjuvant Polymers

Method: Bulk films were cast into 5 cm dia FEP dishes from the solutions using methylene blue as a model drug, RTV 118® as the silicone matrix obtained from GE Silicone, of Waterford, N.Y., and a range PEGs and Pluronic polymers (Ludwigshafen, Germany) as adjuvant polymers. Three molecular weights of PEG were chosen, as were five Pluronic surfactants that varied in molecular weight, physical consistency, and hydrophile-lipophile balance (HLB) (Table 1). Aqueous solutions of adjuvant polymers and methylene blue prepared using a adjuvant polymer:dye mass ratio of 4:1. The solutions were lyophilized and the dry products were ground with a mortar and pestle and sieved to form 180 micron particles. Particles were mixed with 2 g of RTV118 in 8 g anhydrous toluene by shaking. The established silicone/adjuvant polymer/dye mass ratio of 2.0/0.4/0.1 was maintained.

TABLE 1
Adjuvant polymers used for silicone composites.

| | EO | | MW, | |
|---|---|---|---|---|
| | content | Physical form | Da | HLB |
| PEG 1.4k | 100% | Waxy solid | 1450 | (Extrapolated |
| | | | | to 34.6 from |
| | | | | Pluronics data |
| | | | | to 100% EO) |
| PEG 3.4k | 100% | Solid | 3400 | ″ |
| | | | | (Extrapolated |
| | | | | to 34.6 from |
| | | | | Pluronics data |
| | | | | to 100% EO) |
| PEG 20k | 100% | Solid flakes | 20000 | ″ |
| | | | | (Extrapolated |
| | | | | to 34.6 from |
| | | | | Pluronics data |
| | | | | to 100% EO) |
| Pluronic 68 | 80% | Solid | 8400 | 29 |
| | | granule/powder | | |
| Pluronic 88 | 80% | Solid | 11400 | 28 |
| | | granule/powder | | |
| Pluronic | 70% | Solid granules/ | 12600 | 22 |
| 127 Prill | | | | |
| Pluronic | 70% | Solid | 12600 | 22 |
| 127 NR | | granule/powder | | |
| Pluronic | 10% | Liquid | 4400 | 1 |
| 121 | | | | |

Films were allowed to cure for 3 days. 8 mm dia disks were punched from each film, weighed, and soaked in 10 ml PBS at 37° C. with agitation at 300 rpm. At intervals, samples were transferred to fresh PBS and absorbance at 665 nm was measured.

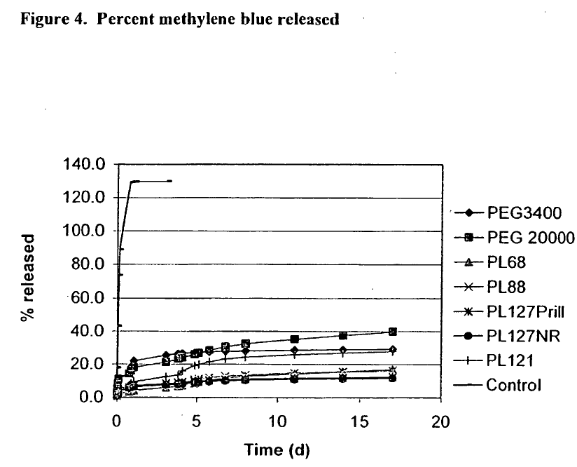

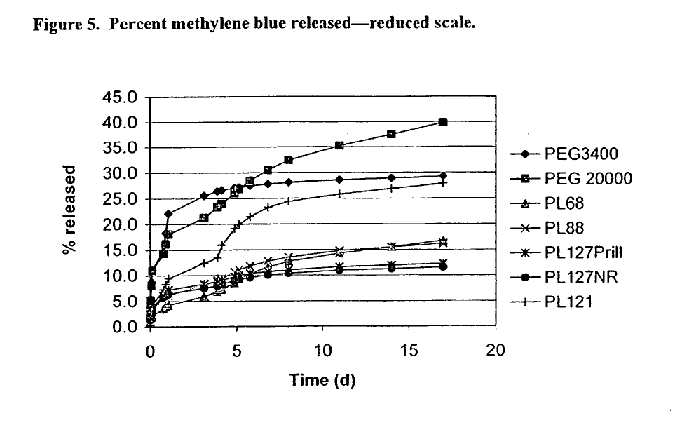

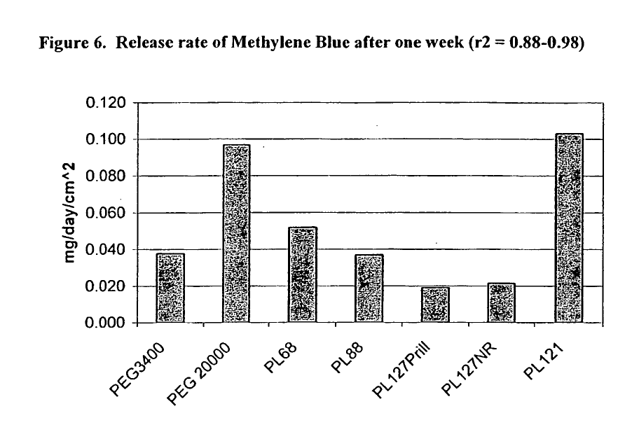

Results FIG. 4 and FIG. 5 indicate that drug release was faster from PEG compatibilized films than from solid Pluronic compatibilized films, suggesting that the presence of the hydrophobic segment of Pluronic slowed diffusion of the hydrophilic drug. There appeared to be a molecular weight dependence to this effect, with diffusion being slower from matrices containing higher molecular weight Pluronic. Although initial release profiles were similar, extended dye release was greater from high molecular weight PEG than low molecular weight PEG. Differences in PEG particle crystallinity as a function of molecular weight could account for this effect. Biphasic release was observed from the matrix containing the very hydrophobic, liquid Pluronic 121 adjuvant polymer, suggesting that the diffusion of water into the composite enhanced the diffusion of dye from the material. After one week, the PEG 20k and PL121 materials sustained high release rates. (FIG. 6). In contrast, the hydrophilic dye burst immediately from the silicone sealant that did not contain hydrophilic adjuvant polymer particles. The burst apparently exceeded the maximum amount of drug calculated to be in the disk. A scan of the dye solution indicated that the peak maxima had not shifted from 665 nm, indicating that the dye had not been modified in any way that would result in an artificially enhanced reading. We attribute the discrepancy to non-uniform aggregation of the dye particles within the silicone film in the absence of a stabilizing adjuvant polymer.

### EXAMPLE 3

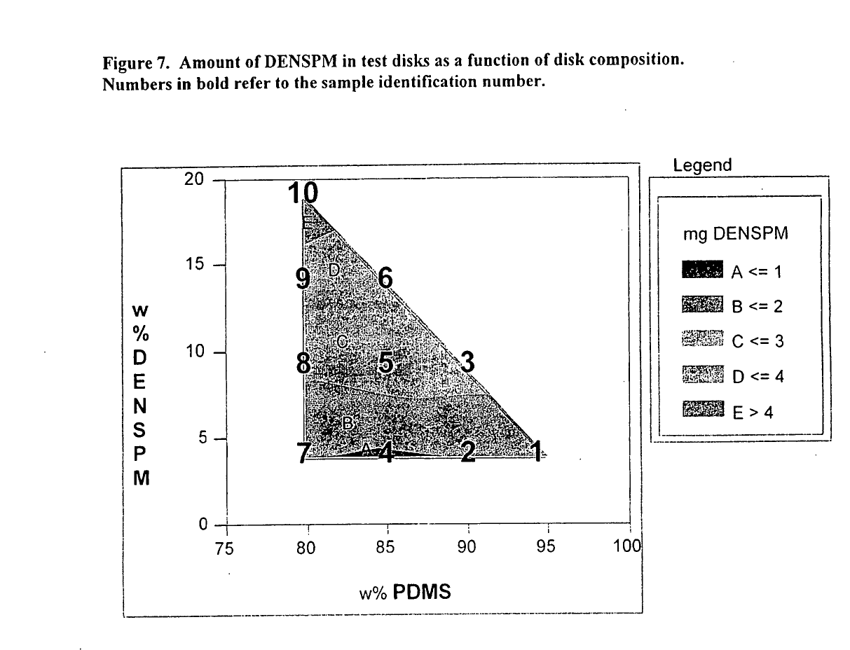

The present invention also provides a silicone matrix containing PEG-drug particles with low drug burst, sustained drug release, and suitable handling and mechanical properties for wrapping around a vein. Toward this end, we have prepared a series of ˜1 mm thick sheets from the polydimethylsiloxane (PDMS), polyethylene glycol, and diethylnorspermine (DENSPM). DENSPM is a hydrophilic polyamine drug chosen for its anti-stenotic properties. The compositions used to prepare the sheets are summarized in Table 2 below and illustrated in FIG. 7. The compositions in the sheets are varied systematically within the following ranges: in PDMS (80-95% wt.), PEG (1-16% wt.) and drug (4-19% wt.).

TABLE 2

| Sample | wt % | wt % | wt % | Modulus | Modulus | Modulus |
|---|---|---|---|---|---|---|
| # | PDMS | PEG | DENSPM | (MPa) | (MPa)^{Y} | (MPa)* |
| 1 | 95 | 1 | 4 | 0.6 | 0.8 | 0.4 |
| 2 | 90 | 6 | 4 | 1.1 | 1.43 | 0.2 |
| 3 | 90 | 1 | 9 | 0.8 | 1.17 | 0.2 |
| 4 | 85 | 11 | 4 | 1.9 | 2.2 | 0.8 |
| 5 | 85 | 6 | 9 | 3.4 | 3.5 | 0.4 |
| 6 | 85 | 1 | 14 | 1.5 | 2.13 | 0.5 |
| 7 | 80 | 16 | 4 | 2.6 | 2.57 | 1.1 |
| 8 | 80 | 11 | 9 | 3.2 | 3.23 | 0.6 |
| 9 | 80 | 6 | 14 | 2.6 | 3.57 | 0.5 |
| 10 | 80 | 1 | 19 | 1.7 | 2.63 | 0.6 |

^{Y}after γ-radiation
*after γ radiation and 2 wk soak

Analysis of the physical characteristics of the sheets is made to determine the maximum amount of drug and minimum amount of PEG that can be loaded without making films that swell, are too friable, or that release drug too quickly (as would be the case for PEG-free controls) such that a drug-release matrix that approximately matches the mechanical characteristics of the vein to be sheathed may be obtained. 10 sheets were prepared and kinetic release study was conducted by monitoring the PH's of the 24 h burst phase.

Method: Samples for Instron testing (4 mm working width) and 6 mm dia. disks were cut from the films and sterilized by gamma irradiation (2.6 MRad).

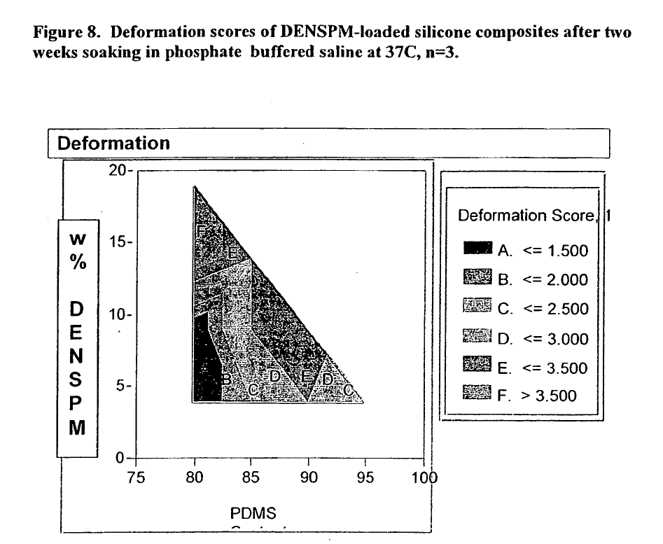

Deformation due to hydration: Instron samples were soaked in 25 ml, 0.2 μm filter sterilized PBS @37° C. for two weeks to assess the ability of films to maintain their shape and strength when hydrated. Afterward, the films were assigned a score from 1-4 based on the degree of deformation as illustrated in FIG. 8. 1 indicates practically flat and 4 indicates completely curled. The films were desiccated (1 atm, room temp, 10-20% relative humidity) for at least three days prior to Instron testing.

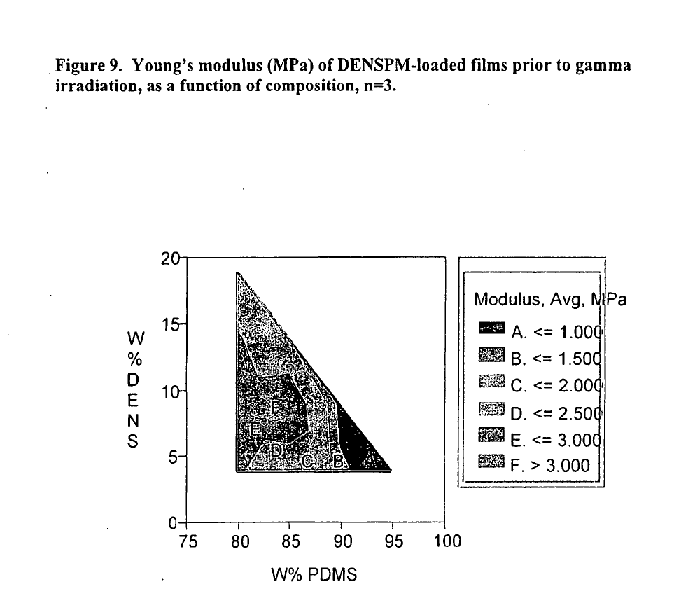

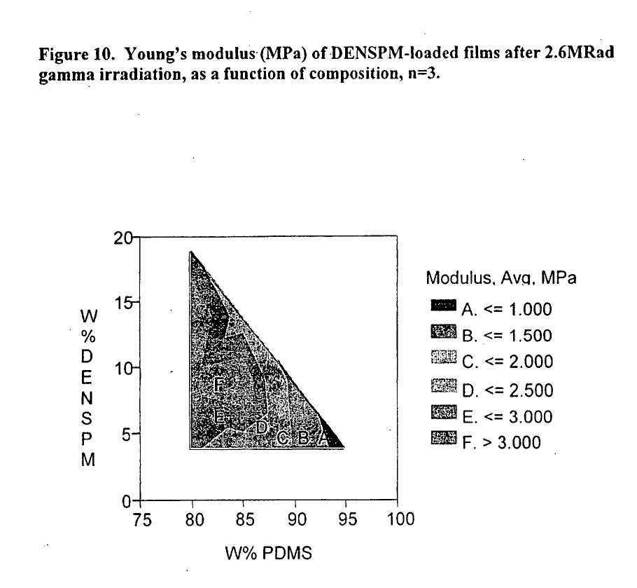

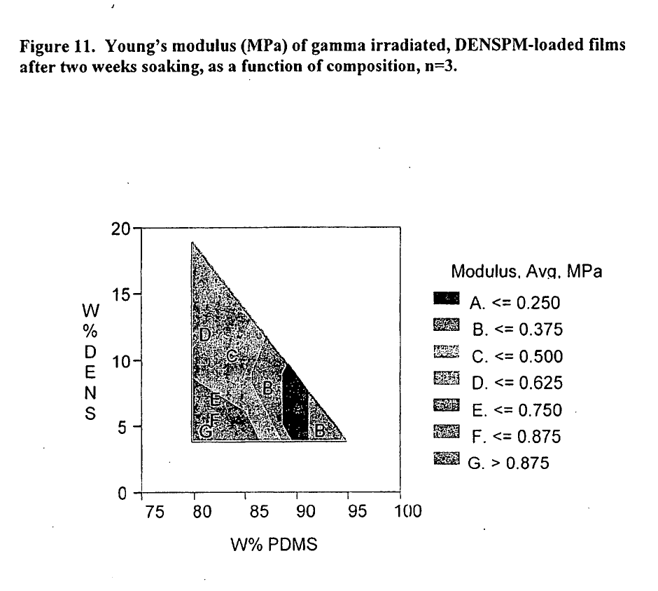

Mechanical testing: Instron testing on each of ten compositions with and without gamma irradiation and from t soaking. 3 additional gamma irradiated samples the she soaking study described above were tested to determine the effect of hydration and drug release on mechanical properties of the matrices. Film thicknesses were measured with digital calipers and films were stretched at 5 cm/min until failure. Modulus, percent elongation at break, and toughness were calculated and compared. For sake of brevity, only modulus is reported in Table 3 and illustrated in FIG. 9 to FIG. 11.

TABLE 3

| Sample | Deformation | Modulus | Modulus | Modulus |
|---|---|---|---|---|
| # | Score | (MPa) | (MPa)^{¥} | (MPa)* |
| 1 | 2 | 0.6 | 0.8 | 0.4 |
| 2 | 3 | 1.1 | 1.43 | 0.2 |
| 3 | 3.5 | 0.8 | 1.17 | 0.2 |
| 4 | 2 | 1.9 | 2.2 | 0.8 |
| 5 | 3 | 3.4 | 3.5 | 0.4 |
| 6 | 3 | 1.5 | 2.13 | 0.5 |
| 7 | 1 | 2.6 | 2.57 | 1.1 |
| 8 | 1 | 3.2 | 3.23 | 0.6 |
| 9 | 4 | 2.6 | 3.57 | 0.5 |
| 10 | 4 | 1.7 | 2.63 | 0.6 |

^{¥}after γ-radiation
*after γ radiation and 2 wk soak

Kinetic Release Study. Three disks of each composition and silicone-only controls were placed in 5 ml 0.2 μm filter sterilized PBS (diluted from 10× concentrate) in 20 ml scintillation vials. The samples were shaken at 300 rpm in a 37° C. box. Samples were transferred to fresh PBS aliquots in a laminar flow hood at 1, 2, 4, 10, 14 and 35 day time points. The pH of aliquots from the 1-day time point was measured and the results are discussed below.

Results:

Deformation Due to Soaking:

The results in FIG. 8 indicate that only sample compositions 7 & 8 did not deform. Compositions 10 & 9 deformed the most. We think that the high content of hydrophilic PEG & DENSPM contributed to anisotropic swelling of the film. The anisotropy may be due to formation of a thin silicone-rich layer on one side of these films due to settling and depletion of PEG-DENSPM particles from the upper surface during curing. It is interesting that at high hydrophilics content (20%), the increasing amount of DENSPM seemed to contribute to anisotropic swelling. Particles with high DENSPM content (>45%) may be denser and more prone to settling, or may not have sufficient PEG to create the desired interfacial interactions with the PDMS environment to keep the particles well suspended during curing and toluene evaporation.

Mechanical Testing:

There is a slight loss in modulus of the films after gamma irradiation (FIGS. 9 & 10). The modulus of the soaked and unsoaked materials generally decreased as silicone content increases. This suggests that the particles are having a filling effect and stiffening the matrices. After soaking and desiccation, the modulus of all samples was reduced The samples that deformed the most (FIG. 8) had the greatest reduction in modulus, perhaps due to hydration and dissolution of the PEG-DENSPM particles and reduction of the filler effect.

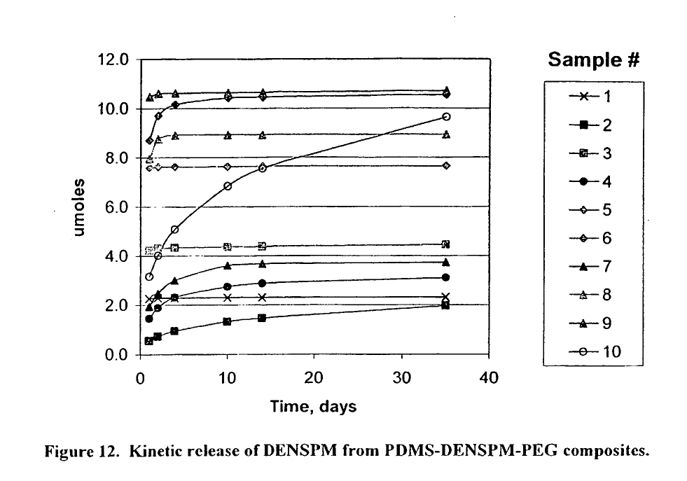

Kinetic release studies. FIG. 12 shows the kinetic release data of DENSPM in 5 ml PBS at 37° C. for various PDMS-DENSPM-PEG compositions. Compositions 2, 4, 7 and 10 all have relatively low bursts and extended release times. In general, release profile is a strong function of PDMS-DENSPM-PEG composition, with bursts occurring in all compositions with greater than 4% DENSPM, except for composition #10, which has the highest DENSPM content, 19%. Release is observed out to 35 days for compositions 2 & 10.

The invention is not limited by the embodiments described above which are presented as examples only but can be modified in various ways within the scope of protection defined by the appended patent claims.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto. All references cited herein are incorporated by reference in their entirety.

I claim:

1. A composition for use in delivering a drug into the body of a mammal, wherein the composition comprises silicone elastomer, an adjuvant polymer, and the drug.

2. The composition of claim 1 wherein the adjuvant polymer is selected from the group consisting of polyethylene glycol or copolymer thereof, polymeric surfactant, polysaccharide, polyurethane, and polyethyleneimines. hyaluronic acid and its chemical derivatives, chemically modified cellulose, polyamyloses, polydextroses, dextrans, heparins, heparans, chondroitin sulfate, dermatan sulfate, poly(N-isopropylacrylamide), polyurethanes, polyacrylates, polyethyleneimines, poly-N-vinylpyrrolidone, polyvinylalcohol, or polyvinylacetate.

3. The composition of claim 2 wherein the polyethylene glycol has a molecular weight of 2-500 kDa

4. The composition of claim 1 wherein the drug is selected from the group consisting of antiproliferative, anti-inflammatory, antibiotic, antiplatelet, anticoagulant, antimicrobial, anti-arrhythmic, antisense, and genetic material.

5. The composition of claim 1 wherein the drug is hydrophilic.

6. The composition of claim 5 wherein the hydrophilic drug is selected from the group consisting of Tranilast, DENSPM, rapamycin and its derivatives thereof.

7. The composition of claim 1 wherein the drug is hydrophobic.

8. The composition of claim 5 wherein the hydrophobic drug is selected from the group consisting of paclitaxel, ciproflaxicin and amiodarone.

9. An implantable medical device comprising a composition that comprises a first polymer of silicone elastomer, an adjuvant polymer, and a drug.

10. The device of claim 9, which is selected from the group consisting of a catheter, a wire guide, a cannula, a stent, a vascular or other graft or sheath, PICC lines, arterial-venous shunt, a cardiac pacemaker lead or lead tip, a cardiac defibrillator lead or lead tip, a heart valve, a suture, or a needle, an angioplasty device or portion thereof, a pacemaker or portion thereof, and an orthopedic device, appliance, implant or replacement.

11. The device of claim 9 comprising a base material comprising a surface, and a first layer applied to at least a portion of the surface comprising said composition.

12. The device of claim 9, wherein the base material comprises stainless steel, tantalum, titanium, nitinol, gold, platinum, inconel, iridium, silver, tungsten, or another biocompatible metal, or alloys of any of these; carbon or carbon fiber; cellulose acetate, cellulose nitrate, silicone, polyethylene teraphthalate, polyurethane, polyamide, polyester, polyorthoester, polyanhydride, polyether sulfone, polycarbonate, polypropylene, polyethylene, polytetrafluoroethylene, or mixtures or copolymers of these, polylactic acid, polyglycolic acid or copolymers thereof, a polyanhydride, polycaprolactone, polyhydroxy-butyrate valerate, or mixtures or copolymers of these.

13. The device of claim 9 wherein the adjuvant polymer is selected from the group consisting of polyethylene glycol, polyethylene glycol containing block copolymers, polymeric surfactant, polysaccharide, polyurethane, and polyethyleneimines.

14. The device of claim 13 wherein the polyethylene glycol has a molecular weight of 2-500 kDa.

15. The device of claim 9 wherein the drug is selected from the group consisting of antiproliferative, anti-inflammatory, antibiotic, antiplatelet, anticoagulant, antimicrobial, anti-arrhythmic, antisense, and genetic material.

16. The device of claim 9 wherein the drug is hydrophilic.

17. The device of claim 16 wherein the hydrophilic drug is selected from the group consisting of Tranilast, DENSPM rapamycin and its derivatives thereof.

18. The device of claim 9 wherein the drug is hydrophobic.

19. The device of claim 18 wherein the hydrophobic drug is selected from the group consisting of paclitaxel, ciproflaxicin and amiodarone.

20. The device of claim 9 wherein the coating layer is further coated with a top layer comprising silicone elastomer.

21. The device of claim 20 wherein the top layer of silicone elastomer further comprises polyethylene glycol.
